(19) United States
(12) Patent Application Publication
(10) Pub. No.: US 2004/0157315 A1
(43) Pub. Date: Aug. 12, 2004

(54) Culture medium for detecting fungi and yeast and method for preparing the medium

(75) Inventor: Mariana Tajada, Buenos Aires (AR)

Correspondence Address: MERCHANT & GOULD PC, P.O. BOX 2903, MINNEAPOLIS, MN, 55402-0903, US

(73) Assignee: Coca-Cola Femsa de Buenos Aires S.A., Buenos Aires (AR)

(21) Appl. No.: 10/731,935

(22) Filed: Dec. 9, 2003

(30) Foreign Application Priority Data
Dec. 10, 2002 (AR) P020104775

(51) Int. Cl. C12N 1/16; C12N 1/18

(52) U.S. Cl. 435/256.8

(57) ABSTRACT

Culture medium composition for fungi and yeast, preparation method and uses of the composition. The culture medium composition is employed for analyzing microorganisms contamination in the food industry, preferably in the cola beverages industry, wherein the composition comprises between 4.5 and 5.5 g/l of monobasic potassium phosphate, between 0.5 and 1.5 g/l of ammonium chloride, between 0.5 and 1.5 g/l of heptahydrate magnesium sulfate, between 30.0 and 50.0 g/l of D(+) saccharose, and water.

### BACKGROUND OF THE INVENTION

1. Field of the Invention

The present invention relates to a culture medium or composition for the culturing, detection, counting and identification of microorganisms and more particularly the invention refers to a culture medium or composition for detecting and counting fungi and/or yeast in any kind of installations, most preferably installations employed in the food industry, with the composition basically comprising a solution of monobasic potassium phosphate, ammonium chloride, heptahydrate magnesium sulfate, D(+) saccharose, and water.

While specific reference will be made in this specification to the application of the inventive medium in the food industry, it will be apparent to any person skilled in the art that the invention can be applied into any field where a control of microorganisms, or any other agent such as undesired or pathogenic agent, is to be carried out.

2. Description of the Prior Art

The food industry, particularly the industry of producing cola beverages, requires of control tests allowing the evaluation of contaminating fungi and yeast that may be present in all and each of the production stages. To date such evaluation is carried out by culturing, in culture mediums, samples taken from the industrial devices or products, and by further analyzing the growing of fungi and/or yeast colonies. To this purpose several culture mediums, such as the Sartorius® dehydrated medium for detecting fungi and yeast and the Bioplast® liquid medium, are employed, the compositions of which mediums being as follows:

Bioplast® liquid medium:

Yeast extract 9.0 g/l±0.5 g/l.

Dextrose 50.0 g/l±0.5 g/l.

Digested pancreatic casein 5.0 g/l±0.5 g/l.

Digested peptides of animal tissue 5.0 g/l±0.5 g/l.

Final pH 4.6±0.2.

Sartorius® dehydrated culture medium:

Yeast extract 9.0 g/l.

Dextrose 50.0 g/l.

Peptone 10.0 g/l.

Magnesium sulfate 2.1 g/l.

Diastase 0.05 g/l.

Thiamine 0.05 g/l.

Final pH 4.6.

In the food or beverages production stages a large number of evaluations must be made to determine the presence of fungi and yeast, this resulting in high costs for the industry using such culture mediums. There is a need, therefore, of having culture medium compositions that would be cheaper but capable of having an efficiency for evaluating colonies that is the same or higher than the culture mediums usually employed in the industry.

### SUMMARY OF THE INVENTION

It is therefore one object of the present invention to provide a culture medium comprising:

i. between about 4.5 g/l and about 5.5 g/l of monobasic potassium phosphate;

ii. between about 0.5 g/l and about 1.5 g/l of ammonium chloride;

iii. between about 0.5 g/l and about 1.5 g/l of heptahydrate magnesium sulfate;

iv. between about 30.0 g/l and about 50.0 g/l of D(+) saccharose, and

v. water, wherein the pH of the medium may be between 4.5 and 5.5.

It is even another object of the present invention to provide a method of preparing a culture medium comprising between about 4.5 g/l and about 5.5 g/l of monobasic potassium phosphate; between about 0.5 g/l and about 1.5 g/l of ammonium chloride; between about 0.5 g/l and about 1.5 g/l of heptahydrate magnesium sulfate; between about 30.0 g/l and about 50.0 g/l of D(+) saccharose, and water, wherein the method comprises the steps of:

a. obtaining a solution by dissolving in ultra pure water, under agitation, the monobasic potassium phosphate, the ammonium chloride, the heptahydrate magnesium sulfate and the saccharose;

b. adjusting the pH of the solution obtained in step a. to 5.0, and

c. sterilizing the solution and conserving the solution at a temperature of 4° C.

It is still another object of the present invention to provide a culture medium composition for detecting fungi and/or yeast in installations, the composition comprising monobasic potassium phosphate, ammonium chloride, heptahydrate magnesium sulfate, D(+) saccharose, and water.

It is a further object of the present invention to provide a culture medium composition for detecting fungi and/or yeast in several installations, preferably food and/or beverages industry installations, wherein the fungi and yeast are selected from the group consisting of Basillus Subtilis, Candida Albicans, Saccharomyces Cerevisiae, Saccharomyces Uvarum, Rhodotorula Rubra, Penicillium Camemberti, Aspergillus Niger, Trychophyton Ajelloi and Geotrichum Candidum.

It is even a further object of the present invention to provide a method of preparing a culture medium comprising monobasic potassium phosphate, ammonium chloride, heptahydrate magnesium sulfate, D(+) saccharose, and water, wherein the method comprises the steps of:

a. obtaining a solution by dissolving in ultra pure water, under agitation, the monobasic potassium phosphate, the ammonium chloride, the heptahydrate magnesium sulfate and the saccharose;

b. adjusting the pH of the solution obtained in step a. to 5.0, and

c. sterilizing the solution and conserving the solution at a temperature of 4° C.

### BRIEF DESCRIPTION OF THE DRAWINGS

The present invention is illustrated by way of example in the following drawings wherein:

FIG. 1a is a photo showing the development of microorganisms colonies of one sample after 3 days of culturing by employing the culture medium composition of the invention, and

FIG. 1b is a photo showing the development of microorganisms colonies of one sample after 3 days of culturing by employing the culture medium Sartorius®.

### DESCRIPTION OF THE PREFERRED EMBODIMENTS

The inventive composition may be employed for evaluating, in the food industry, the presence of contaminating fungi and yeast. In a preferred embodiment the composition of the invention is employed for growing and counting fungi and yeast colonies in the cola beverages industry, for instance by taking samples in each of the production stages or by taking samples from the products employed in the process for elaborating the beverages.

Any expert in the art will understand that the culture medium composition of the invention may be used for culturing a variety of organisms, such as fungi and yeast, in a number of different industrial processes including processes for elaborating beverages, food products and other products where the presence of said organisms must be kept under control.

With the purpose of testing the effectiveness of the inventive composition as compared to other commercial culture mediums, a number of fungi and yeast cultures have been prepared from different dilutions of a mother solution containing microorganisms obtained from isolated colonies.

The test results are shown in the following Table:

TABLE 1

| | | | Composition |
|---|---|---|---|
| | | | of the |
| | Sartorius ® | Bioplast ® | invention |
| Target | 0 ufc | 0 ufc | 0 ufc |
| Concentrated | TNTC | TNTC | TNTC |
| 1/10² | TNTC | TNTC | TNTC |
| 1/10⁴ | 25 ufc | TNTC | TNTC |
| 1/10⁶ | 0 ufc | 106 ufc | 109 ufc |
| 1/10⁸ | 0 ufc | 4 ufc | 6 ufc |
| 1/10¹⁰ | 0 ufc | 0 ufc | 0 ufc |
| 1/10¹² | 0 ufc | 0 ufc | 0 ufc |
| 1/10¹⁴ | 0 ufc | 0 ufc | 0 ufc |
| 1/10¹⁶ | 0 ufc | 0 ufc | 0 ufc |
| 1/10¹⁸ | 0 ufc | 0 ufc | 0 ufc |
| 1/10²⁰ | 0 ufc | 0 ufc | 0 ufc |

To the purpose of the present specification the term “TNTC” means “too much colonies to be counted”.

As may be seen from the above Table the inventive composition complied with exigent requirements for the culturing tests of microorganisms present in the cola beverages production plants, thus permitting the proper growing not only of fungi but also of yeast, and also providing a proper medium insofar as the nutrients availability, humidity and pH.

While the medium Sartorius® did not provide a response to samples having a very low contamination, the culture medium Bioplast® and the composition of the invention did not have obstacles in evidencing the contamination that was present in the solution at low concentrations.

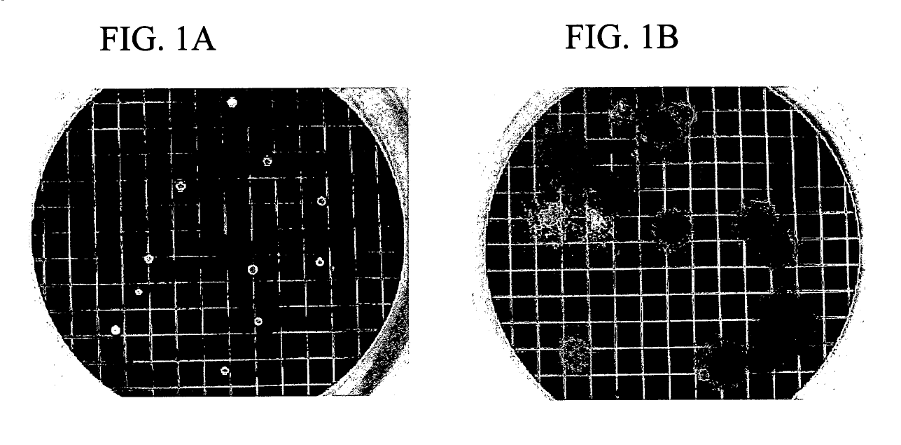

It was also observed that the colonies grown in the inventive medium had dimensions smaller than the ones of the other mediums; this feature permitted to more easily count the colonies in high contamination levels because the colonies did not form an homogeneous layer on the culture membrane (FIGS. 1a and 1b).

The colonies counted in the analyzed plates were similar and almost the same for three tests.

Before carrying out the above mentioned tests, a number of evaluations consisting of comparing the culture mediums Sartorius®, Bioplast® and the one of the invention have been made with samples taken from filling machines in the production lines, thus guaranteeing in a larger extent the efficiency and quality of the inventive culture medium composition.

The tests results are shown in the following Table.

TABLE 2

| | | Composition |
|---|---|---|
| | Medium | of the |
| | Sartorius ® | invention |
| Valve n° 15 | 00 ufc | 04 ufc |
| Valve n° 30 | 00 ufc | 08 ufc |
| Valve n° 45 | 00 ufc | 05 ufc |
| Valve n° 60 | 00 ufc | 07 ufc |

It is apparent from the above results that the culture composition of the invention provides a response that is better than the one of the commercial medium in the detection of microorganisms when the same are present in a very low concentration.

When the composition of the invention has been compared to the commercial culture mediums Sartorius® and Bioplast® in connection to the response to high and low concentrations of fungi the following results have been found:

TABLE 3

| | Contami- | | | |
|---|---|---|---|---|
| Sample | nation | Culture Medium | | |
| No. | degree | Sartorius ® | Bioplast ® | Invention |
| 1 | High | TNTC | TNTC | TNTC |
| 2 | High | TNTC | TNTC | TNTC |
| 3 | High | TNTC | TNTC | TNTC |
| 4 | High | TNTC | TNTC | TNTC |
| 5 | High | TNTC | TNTC | TNTC |
| 6 | High | TNTC | TNTC | TNTC |
| 7 | Low | 05 ufc | 04 ufc | 04 ufc |

From the results in the above Table 3 it may be seen that either in samples with low microbial concentrations as well as in samples with excessive contamination the inventive composition provides a response similar to the other tested mediums, comparatively giving an equivalent amount of ufc.

A particular feature of this new culture medium composition is the size that the colonies have when growing in the inventive medium. For contaminations having values of about 300-400 ufc, the colonies developed in mediums Sartorius® and Bioplast® have a considerable size or dimension producing a homogeneous and continuous layer formed in the medium thus preventing the colonies from being counted. For the same amount of colonies the composition of the invention allows a correct counting of colonies because the colonies are of smaller size and are spaced apart from each other.

The above mentioned test results show that the composition of the invention is effective for the counting of fungi and yeast commonly found in the food industry and more preferably for the counting of fungi and yeast that are present in the production lines of cola beverages.

The 500 comparative tests in the following Table show that the composition of the invention has a fungi and yeast detection level that is equal or superior to the one of available commercial culture mediums.

TABLE 4

| Test | | | | Sample | Inventive | Bioplast | Sartorius |
|---|---|---|---|---|---|---|---|
| # | Date | Sector | Equipment | Type | Composition | ® | ® |
| 1 | 8 Nov. 2001 | Unloading | Transport | Fructose | 01 ufc | — | 00 ufc |
| 2 | 8 Nov. 2001 | Unloading | Transport | Fructose | 00 ufc | — | 01 ufc |
| 3 | 8 Nov. 2001 | Unloading | Transport | Fructose | 02 ufc | — | 03 ufc |
| 4 | 8 Nov. 2001 | Unloading | Transport | Fructose | 02 ufc | — | 02 ufc |
| 5 | 8 Nov. 2001 | Unloading | Transport | Fructose | 01 ufc | — | 01 ufc |
| 6 | 8 Nov. 2001 | Unloading | Transport | Fructose | 00 ufc | — | 02 ufc |
| 7 | 9 Nov. 2001 | Bottling | Filling | Sanitation | 05 ufc | — | 08 ufc |
| | | | Valve | Water | | | |
| 8 | 9 Nov. 2001 | Bottling | Filling | Sanitation | 02 ufc | — | 06 ufc |
| | | | Valve | Water | | | |
| 9 | 9 Nov. 2001 | Bottling | Filling | Sanitation | 10 ufc | — | 03 ufc |
| | | | Valve | Water | | | |
| 10 | 9 Nov. 2001 | Bottling | Filling | Sanitation | 07 ufc | — | 09 ufc |
| | | | Valve | Water | | | |
| 11 | 10 Nov. 2001 | Bottling | Filling | Sanitation | 10 ufc | — | 09 ufc |
| | | | Valve | Water | | | |
| 12 | 10 Nov. 2001 | Bottling | Filling | Sanitation | 08 ufc | — | 07 ufc |
| | | | Valve | Water | | | |
| 13 | 10 Nov. 2001 | Bottling | Filling | Sanitation | 08 ufc | — | 05 ufc |
| | | | Valve | Water | | | |
| 14 | 10 Nov. 2001 | Bottling | Filling | Sanitation | 11 ufc | — | 13 ufc |
| | | | Valve | Water | | | |
| 15 | 11 Nov. 2001 | Quality | Laboratory | Prepared | 23 ufc | — | 87 ufc |
| | | Control | | | | | |
| 16 | 11 Nov. 2001 | Quality | Laboratory | Prepared | 62 ufc | — | 117 ufc |
| | | Control | | | | | |
| 17 | 11 Nov. 2001 | Quality | Laboratory | Prepared | 56 ufc | — | 99 ufc |
| | | Control | | | | | |
| 18 | 11 Nov. 2001 | Quality | Laboratory | Prepared | 26 ufc | — | 88 ufc |
| | | Control | | | | | |
| 19 | 11 Nov. 2001 | Quality | Laboratory | Prepared | 54 ufc | — | 83 ufc |
| | | Control | | | | | |
| 20 | 20 Nov. 2001 | Quality | Laboratory | Prepared | TNTC | TNTC | TNTC |
| | | Control | | | | | |
| 21 | 20 Nov. 2001 | Quality | Laboratory | Prepared | TNTC | TNTC | TNTC |
| | | Control | | | | | |
| 22 | 20 Nov. 2001 | Quality | Laboratory | Prepared | TNTC | TNTC | TNTC |
| | | Control | | | | | |
| 23 | 20 Nov. 2001 | Quality | Laboratory | Prepared | TNTC | TNTC | TNTC |
| | | Control | | | | | |
| 24 | 20 Nov. 2001 | Quality | Laboratory | Prepared | TNTC | TNTC | TNTC |
| | | Control | | | | | |
| 25 | 21 Nov. 2001 | Quality | Laboratory | Prepared | TNTC | TNTC | TNTC |
| | | Control | | | | | |
| 26 | 21 Nov. 2001 | Quality | Laboratory | Prepared | TNTC | TNTC | TNTC |
| | | Control | | | | | |
| 27 | 21 Nov. 2001 | Quality | Laboratory | Prepared | TNTC | TNTC | TNTC |
| | | Control | | | | | |
| 28 | 21 Nov. 2001 | Quality | Laboratory | Prepared | TNTC | TNTC | TNTC |
| | | Control | | | | | |
| 29 | 21 Nov. 2001 | Quality | Laboratory | Prepared | TNTC | TNTC | TNTC |
| | | Control | | | | | |
| 30 | 23 Nov. 2001 | Bottling | Filling | Sanitation | 01 ufc | — | 01 ufc |
| | | | Valve | Water | | | |
| 31 | 23 Nov. 2001 | Bottling | Filling | Sanitation | 00 ufc | — | 03 ufc |
| | | | Valve | Water | | | |
| 32 | 23 Nov. 2001 | Bottling | Filling | Sanitation | 01 ufc | — | 01 ufc |
| | | | Valve | Water | | | |
| 33 | 24 Nov. 2001 | Bottling | Filling | Sanitation | 00 ufc | — | 00 ufc |
| | | | Valve | Water | | | |
| 34 | 24 Nov. 2001 | Bottling | Filling | Sanitation | 00 ufc | — | 00 ufc |
| | | | Valve | Water | | | |
| 35 | 24 Nov. 2001 | Bottling | Filling | Sanitation | 00 ufc | | 00 ufc |
| | | | Valve | Water | | | |
| 36 | 26 Nov. 2001 | Quality | Laboratory | Prepared | TNTC | TNTC | TNTC |
| | | Control | | | | | |
| 37 | 26 Nov. 2001 | Quality | Laboratory | Prepared | TNTC | TNTC | TNTC |
| | | Control | | | | | |
| 38 | 26 Nov. 2001 | Quality | Laboratory | Prepared | TNTC | TNTC | TNTC |
| | | Control | | | | | |
| 39 | 26 Nov. 2001 | Quality | Laboratory | Prepared | TNTC | TNTC | TNTC |
| | | Control | | | | | |
| 40 | 26 Nov. 2001 | Quality | Laboratory | Prepared | TNTC | TNTC | TNTC |
| | | Control | | | | | |
| 41 | 28 Nov. 2001 | Quality | Laboratory | Prepared | TNTC | TNTC | TNTC |
| | | Control | | | | | |
| 42 | 28 Nov. 2001 | Quality | Laboratory | Prepared | TNTC | TNTC | TNTC |
| | | Control | | | | | |
| 43 | 28 Nov. 2001 | Quality | Laboratory | Prepared | TNTC | TNTC | TNTC |
| | | Control | | | | | |
| 44 | 28 Nov. 2001 | Quality | Laboratory | Prepared | TNTC | TNTC | TNTC |
| | | Control | | | | | |
| 45 | 28 Nov. 2001 | Quality | Laboratory | Prepared | TNTC | TNTC | TNTC |
| | | Control | | | | | |
| 46 | 29 Nov. 2001 | Bottling | Filling | Sanitation | 00 ufc | 00 ufc | 00 ufc |
| | | | Valve | Water | | | |
| 47 | 29 Nov. 2001 | Bottling | Filling | Sanitation | 00 ufc | 00 ufc | 00 ufc |
| | | | Valve | Water | | | |
| 48 | 29 Nov. 2001 | Bottling | Filling | Sanitation | 00 ufc | 00 ufc | 00 ufc |
| | | | Valve | Water | | | |
| 49 | 29 Nov. 2001 | Bottling | Filling | Sanitation | 00 ufc | 00 ufc | 00 ufc |
| | | | Valve | Water | | | |
| 50 | 29 Nov. 2001 | Bottling | Filling | Sanitation | 00 ufc | 00 ufc | 00 ufc |
| | | | Valve | Water | | | |
| 51 | 28 Jan. 2002 | Bottling | Filling | Sanitation | 04 ufc | — | 00 ufc |
| | | | Valve | Water | | | |
| 52 | 28 Jan. 2002 | Bottling | Filling | Sanitation | 08 ufc | — | 01 ufc |
| | | | Valve | Water | | | |
| 53 | 28 Jan. 2002 | Bottling | Filling | Sanitation | 05 ufc | — | 01 ufc |
| | | | Valve | Water | | | |
| 54 | 28 Jan. 2002 | Bottling | Filling | Sanitation | 07 ufc | — | 00 ufc |
| | | | Valve | Water | | | |
| 55 | 4 Mar. 2002 | Quality | Laboratory | Prepared | TNTC | — | TNTC |
| | | Control | | | | | |
| 56 | 4 Mar. 2002 | Quality | Laboratory | Prepared | TNTC | — | TNTC |
| | | Control | | | | | |
| 57 | 4 Mar. 2002 | Quality | Laboratory | Prepared | TNTC | — | TNTC |
| | | Control | | | | | |
| 58 | 4 Mar. 2002 | Quality | Laboratory | Prepared | TNTC | — | TNTC |
| | | Control | | | | | |
| 59 | 4 Mar. 2002 | Quality | Laboratory | Prepared | TNTC | — | TNTC |
| | | Control | | | | | |
| 60 | 4 Mar. 2002 | Quality | Laboratory | Prepared | TNTC | — | TNTC |
| | | Control | | | | | |
| 61 | 4 Mar. 2002 | Quality | Laboratory | Prepared | TNTC | — | TNTC |
| | | Control | | | | | |
| 62 | 4 Mar. 2002 | Quality | Laboratory | Prepared | TNTC | — | TNTC |
| | | Control | | | | | |
| 63 | 4 Mar. 2002 | Quality | Laboratory | Prepared | TNTC | — | TNTC |
| | | Control | | | | | |
| 64 | 4 Mar. 2002 | Quality | Laboratory | Prepared | TNTC | — | TNTC |
| | | Control | | | | | |
| 65 | 8 Feb. 2002 | Quality | Laboratory | Prepared | TNTC | TNTC | — |
| | | Control | | | | | |
| 66 | 8 Feb. 2002 | Quality | Laboratory | Prepared | TNTC | TNTC | — |
| | | Control | | | | | |
| 67 | 8 Feb. 2002 | Quality | Laboratory | Prepared | TNTC | TNTC | — |
| | | Control | | | | | |
| 68 | 8 Feb. 2002 | Quality | Laboratory | Prepared | TNTC | TNTC | — |
| | | Control | | | | | |
| 69 | 8 Feb. 2002 | Quality | Laboratory | Prepared | TNTC | TNTC | — |
| | | Control | | | | | |
| 70 | 8 Feb. 2002 | Quality | Laboratory | Prepared | TNTC | TNTC | — |
| | | Control | | | | | |
| 71 | 8 Feb. 2002 | Quality | Laboratory | Prepared | TNTC | TNTC | — |
| | | Control | | | | | |
| 72 | 8 Feb. 2002 | Quality | Laboratory | Prepared | TNTC | TNTC | — |
| | | Control | | | | | |
| 73 | 8 Feb. 2002 | Quality | Laboratory | Prepared | TNTC | TNTC | — |
| | | Control | | | | | |
| 74 | 8 Feb. 2002 | Quality | Laboratory | Prepared | TNTC | TNTC | — |
| | | Control | | | | | |
| 75 | 19 Mar. 2001 | Quality | Laboratory | Common | 19 ufc | 17 ufc | — |
| | | Control | | Water | | | |
| 76 | 19 Mar. 2001 | Quality | Laboratory | Common | 26 ufc | 23 ufc | — |
| | | Control | | Water | | | |
| 77 | 19 Mar. 2001 | Quality | Laboratory | Common | 17 ufc | 20 ufc | — |
| | | Control | | Water | | | |
| 78 | 19 Mar. 2001 | Quality | Laboratory | Common | 23 ufc | 14 ufc | — |
| | | Control | | Water | | | |
| 79 | 19 Mar. 2001 | Quality | Laboratory | Common | 20 ufc | 16 ufc | — |
| | | Control | | Water | | | |
| 80 | 19 Mar. 2001 | Quality | Laboratory | Common | 24 ufc | 25 ufc | — |
| | | Control | | Water | | | |
| 81 | 26 Mar. 2002 | Water Sector | Tank 1050 | Raw | 01 ufc | 01 ufc | — |
| | | | | Water | | | |
| 82 | 26 Mar. 2002 | Quality | Laboratory | Water | 00 ufc | 00 ufc | — |
| | | Control | | | | | |
| 83 | 26 Mar. 2002 | Quality | Laboratory | Common | 01 ufc | 02 ufc | — |
| | | Control | | Water | | | |
| 84 | 26 Mar. 2002 | Water Room | Outlet FP1 | Treated | 02 ufc | 01 ufc | — |
| | | | | Water | | | |
| 85 | 26 Mar. 2002 | Water Room | Tank 600 | Chlorinated | 00 ufc | 00 ufc | — |
| | | | | Water | | | |
| 86 | 27 Mar. 2002 | Water Room | Outlet FP1 | Treated | 00 ufc | 00 ufc | — |
| | | | | Water | | | |
| 87 | 27 Mar. 2002 | Bottling | Filling | Sanitation | 02 ufc | 01 ufc | — |
| | | | Valve | Water | | | |
| 88 | 27 Mar. 2002 | Bottling | Filling | Sanitation | 01 ufc | 01 ufc | — |
| | | | Valve | Water | | | |
| 89 | 27 Mar. 2002 | Bottling | Filling | Sanitation | 00 ufc | 00 ufc | — |
| | | | Valve | Water | | | |
| 90 | 27 Mar. 2002 | Bottling | Filling | Sanitation | 00 ufc | 00 ufc | — |
| | | | Valve | Water | | | |
| 91 | 27 Mar. 2002 | Bottling | Filling | Sanitation | 05 ufc | 03 ufc | — |
| | | | Valve | Water | | | |
| 92 | 27 Mar. 2002 | Bottling | Filling | Sanitation | 03 ufc | 04 ufc | — |
| | | | Valve | Water | | | |
| 93 | 27 Mar. 2002 | Bottling | Filling | Sanitation | 00 ufc | 00 ufc | — |
| | | | Valve | Water | | | |
| 94 | 27 Mar. 2002 | Bottling | Filling | Sanitation | 00 ufc | 00 ufc | — |
| | | | Valve | Water | | | |
| 95 | 27 Mar. 2002 | Bottling | Filling | Sanitation | 00 ufc | 00 ufc | — |
| | | | Valve | Water | | | |
| 96 | 27 Mar. 2002 | Bottling | Filling | Sanitation | 00 ufc | 00 ufc | — |
| | | | Valve | Water | | | |
| 97 | 27 Mar. 2002 | Bottling | Filling | Sanitation | 00 ufc | 01 ufc | — |
| | | | Valve | Water | | | |
| 98 | 27 Mar. 2002 | Bottling | Filling | Sanitation | 01 ufc | 01 ufc | — |
| | | | Valve | Water | | | |
| 99 | 27 Mar. 2002 | Bottling | Filling | Sanitation | 00 ufc | 00 ufc | — |
| | | | Valve | Water | | | |
| 100 | 27 Mar. 2002 | Bottling | Filling | Sanitation | 03 ufc | 01 ufc | — |
| | | | Valve | Water | | | |
| 101 | 27 Mar. 2002 | Bottling | Filling | Sanitation | 05 ufc | 02 ufc | — |
| | | | Valve | Water | | | |
| 102 | 27 Mar. 2002 | Bottling | Filling | Sanitation | 02 ufc | 01 ufc | — |
| | | | Valve | Water | | | |
| 103 | 27 Mar. 2002 | Bottling | Filling | Sanitation | 02 ufc | 03 ufc | — |
| | | | Valve | Water | | | |
| 104 | 27 Mar. 2002 | Bottling | Filling | Sanitation | 00 ufc | 00 ufc | — |
| | | | Valve | Water | | | |
| 105 | 27 Mar. 2002 | Bottling | Filling | Sanitation | 01 ufc | 00 ufc | — |
| | | | Valve | Water | | | |
| 106 | 27 Mar. 2002 | Bottling | Filling | Sanitation | 00 ufc | 00 ufc | — |
| | | | Valve | Water | | | |
| 107 | 27 Mar. 2002 | Bottling | Filling | Sanitation | 00 ufc | 00 ufc | — |
| | | | Valve | Water | | | |
| 108 | 27 Mar. 2002 | Bottling | Filling | Sanitation | 02 ufc | 00 ufc | — |
| | | | Valve | Water | | | |
| 109 | 27 Mar. 2002 | Bottling | Filling | Sanitation | 03 ufc | 02 ufc | — |
| | | | Valve | Water | | | |
| 110 | 27 Mar. 2002 | Bottling | Filling | Sanitation | 00 ufc | 01 ufc | — |
| | | | Valve | Water | | | |
| 111 | 27 Mar. 2002 | Bottling | Filling | Sanitation | 00 ufc | 01 ufc | — |
| | | | Valve | Water | | | |
| 112 | 27 Mar. 2002 | Bottling | Filling | Sanitation | 00 ufc | 00 ufc | — |
| | | | Valve | Water | | | |
| 113 | 27 Mar. 2002 | Bottling | Filling | Sanitation | 00 ufc | 00 ufc | — |
| | | | Valve | Water | | | |
| 114 | 27 Mar. 2002 | Bottling | Filling | Sanitation | 01 ufc | 00 ufc | — |
| | | | Valve | Water | | | |
| 115 | 27 Mar. 2002 | Bottling | Filling | Sanitation | 00 ufc | 00 ufc | — |
| | | | Valve | Water | | | |
| 116 | 27 Mar. 2002 | Bottling | Filling | Sanitation | 00 ufc | 00 ufc | — |
| | | | Valve | Water | | | |
| 117 | 27 Mar. 2002 | Bottling | Filling | Sanitation | 02 ufc | 02 ufc | — |
| | | | Valve | Water | | | |
| 118 | 27 Mar. 2002 | Bottling | Filling | Sanitation | 01 ufc | 00 ufc | — |
| | | | Valve | Water | | | |
| 119 | 27 Mar. 2002 | Bottling | Tank JT | Sanitation | 00 ufc | 02 ufc | — |
| | | | | Water | | | |
| 120 | 27 Mar. 2002 | Bottling | Tank JT | Sanitation | 07 ufc | 05 ufc | — |
| | | | | Water | | | |
| 121 | 27 Mar. 2002 | Bottling | Tank JT | Sanitation | 02 ufc | 03 ufc | — |
| | | | | Water | | | |
| 122 | 27 Mar. 2002 | Bottling | Tank JT | Sanitation | 00 ufc | 00 ufc | — |
| | | | | Water | | | |
| 123 | 27 Mar. 2002 | Bottling | Tank JT | Sanitation | 02 ufc | 01 ufc | — |
| | | | | Water | | | |
| 124 | 27 Mar. 2002 | Bottling | Tank JT | Sanitation | 02 ufc | 01 ufc | — |
| | | | | Water | | | |
| 125 | 27 Mar. 2002 | Bottling | Tank JT | Sanitation | 00 ufc | 01 ufc | — |
| | | | | Water | | | |
| 126 | 27 Mar. 2002 | Bottling | Tank JT | Sanitation | 03 ufc | 01 ufc | — |
| | | | | Water | | | |
| 127 | 27 Mar. 2002 | Bottling | Beverage | Sanitation | 03 ufc | 00 ufc | — |
| | | | Tank | Water | | | |
| 128 | 27 Mar. 2002 | Bottling | Beverage | Sanitation | 02 ufc | 01 ufc | — |
| | | | Tank | Water | | | |
| 129 | 27 Mar. 2002 | Bottling | Beverage | Sanitation | 00 ufc | 00 ufc | — |
| | | | Tank | Water | | | |
| 130 | 27 Mar. 2002 | Bottling | Beverage | Sanitation | 01 ufc | 00 ufc | — |
| | | | Tank | Water | | | |
| 131 | 27 Mar. 2002 | Bottling | Beverage | Sanitation | 02 ufc | 04 ufc | — |
| | | | Tank | Water | | | |
| 132 | 27 Mar. 2002 | Bottling | Beverage | Sanitation | 01 ufc | 02 ufc | — |
| | | | Tank | Water | | | |
| 133 | 27 Mar. 2002 | Bottling | Beverage | Sanitation | 00 ufc | 00 ufc | — |
| | | | Tank | Water | | | |
| 134 | 27 Mar. 2002 | Bottling | Beverage | Sanitation | 02 ufc | 00 ufc | — |
| | | | Tank | Water | | | |
| 135 | 27 Mar. 2002 | Bottling | Final | Schweppes | 00 ufc | 00 ufc | — |
| | | | Product | Tonic | | | |
| 136 | 27 Mar. 2002 | Bottling | Final | Schweppes | 00 ufc | 01 ufc | — |
| | | | Product | Tonic | | | |
| 137 | 27 Mar. 2002 | Bottling | Final | Schweppes | 00 ufc | 00 ufc | — |
| | | | Product | Tonic | | | |
| 138 | 27 Mar. 2002 | Bottling | Final | Schweppes | 01 ufc | 00 ufc | — |
| | | | Product | Tonic | | | |
| 139 | 27 Mar. 2002 | Bottling | Final | Schweppes | 00 ufc | 00 ufc | — |
| | | | Product | Tonic | | | |
| 140 | 27 Mar. 2002 | Bottling | Final | Schweppes | 00 ufc | 00 ufc | — |
| | | | Product | Tonic | | | |
| 141 | 27 Mar. 2002 | Bottling | Final | Schweppes | 02 ufc | 00 ufc | — |
| | | | Product | Citrus | | | |
| 142 | 27 Mar. 2002 | Bottling | Final | Schweppes | 00 ufc | 00 ufc | — |
| | | | Product | Citrus | | | |
| 143 | 27 Mar. 2002 | Bottling | Final | Schweppes | 00 ufc | 00 ufc | — |
| | | | Product | Citrus | | | |
| 144 | 27 Mar. 2002 | Bottling | Final | Schweppes | 00 ufc | 00 ufc | — |
| | | | Product | Citrus | | | |
| 145 | 27 Mar. 2002 | Bottling | Final | Schweppes | 00 ufc | 01 ufc | — |
| | | | Product | Citrus | | | |
| 146 | 27 Mar. 2002 | Bottling | Final | Schweppes | 00 ufc | 01 ufc | — |
| | | | Product | Citrus | | | |
| 147 | 27 Mar. 2002 | Bottling | Final | Schweppes | 00 ufc | 00 ufc | — |
| | | | Product | Ginger Ale | | | |
| 148 | 27 Mar. 2002 | Bottling | Final | Schweppes | 00 ufc | 00 ufc | — |
| | | | Product | Ginger Ale | | | |
| 149 | 27 Mar. 2002 | Bottling | Final | Schweppes | 00 ufc | 00 ufc | — |
| | | | Product | Ginger Ale | | | |
| 150 | 27 Mar. 2002 | Bottling | Final | Schweppes | 00 ufc | 02 ufc | — |
| | | | Product | Ginger Ale | | | |
| 151 | 27 Mar. 2002 | Bottling | Final | Schweppes | 00 ufc | 00 ufc | — |
| | | | Product | Ginger Ale | | | |
| 152 | 27 Mar. 2002 | Bottling | Final | Schweppes | 00 ufc | 00 ufc | — |
| | | | Product | Ginger Ale | | | |
| 153 | 27 Mar. 2002 | Bottling | Final | Tai Pink | 00 ufc | 01 ufc | — |
| | | | Product | Grapefruit | | | |
| 154 | 27 Mar. 2002 | Bottling | Final | Tai Pink | 00 ufc | 00 ufc | — |
| | | | Product | Grapefruit | | | |
| 155 | 27 Mar. 2002 | Bottling | Final | Tai Pink | 00 ufc | 00 ufc | — |
| | | | Product | Grapefruit | | | |
| 156 | 27 Mar. 2002 | Bottling | Final | Tai Pink | 00 ufc | 00 ufc | — |
| | | | Product | Grapefruit | | | |
| 157 | 27 Mar. 2002 | Bottling | Final | Tai Pink | 00 ufc | 00 ufc | — |
| | | | Product | Grapefruit | | | |
| 158 | 27 Mar. 2002 | Bottling | Final | Tai Pink | 00 ufc | 00 ufc | — |
| | | | Product | Grapefruit | | | |
| 159 | 27 Mar. 2002 | Syrup | Contimol | Sanitation | 00 ufc | 00 ufc | — |
| | | Room | | Water | | | |
| 160 | 27 Mar. 2002 | Syrup | Pasteurizer | Sanitation | 01 ufc | 00 ufc | — |
| | | Room | | Water | | | |
| 161 | 27 Mar. 2002 | Syrup | Filter TLG | Sanitation | 00 ufc | 00 ufc | — |
| | | Room | | Water | | | |
| 162 | 27 Mar. 2002 | Syrup | Tank Buffer | Sanitation | 03 ufc | 02 ufc | — |
| | | Room | | Water | | | |
| 163 | 27 Mar. 2002 | Syrup | Gravimol 1 | Sanitation | 02 ufc | 01 ufc | — |
| | | Room | | Water | | | |
| 164 | 27 Mar. 2002 | Syrup | Gravimol 1 | Sanitation | 05 ufc | 02 ufc | — |
| | | Room | | Water | | | |
| 165 | 27 Mar. 2002 | Syrup | Gravimol 1 | Sanitation | 06 ufc | 05 ufc | — |
| | | Room | | Water | | | |
| 166 | 27 Mar. 2002 | Syrup | Gravimol 1 | Sanitation | 09 ufc | 06 ufc | — |
| | | Room | | Water | | | |
| 167 | 27 Mar. 2002 | Syrup | Gravimol 1 | Sanitation | 01 ufc | 00 ufc | — |
| | | Room | | Water | | | |
| 168 | 27 Mar. 2002 | Syrup | Tank JS 1 | Sanitation | 00 ufc | 00 ufc | — |
| | | Room | | Water | | | |
| 169 | 27 Mar. 2002 | Syrup | Tank JT 10 | Sanitation | 00 ufc | 00 ufc | — |
| | | Room | | Water | | | |
| 170 | 27 Mar. 2002 | Syrup | Tank JT 5 | Sanitation | 01 ufc | 00 ufc | — |
| | | Room | | Water | | | |
| 171 | 27 Mar. 2002 | Syrup | Tank JT 4 | Sanitation | 02 ufc | 00 ufc | — |
| | | Room | | Water | | | |
| 172 | 27 Mar. 2002 | Syrup | Tank JT 2 | Sanitation | 04 ufc | 05 ufc | — |
| | | Room | | Water | | | |
| 173 | 27 Mar. 2002 | Syrup | Tank JT 1 | Sanitation | 00 ufc | 02 ufc | — |
| | | Room | | Water | | | |
| 174 | 27 Mar. 2002 | Syrup | Tank JT 6 | Sanitation | 03 ufc | 00 ufc | — |
| | | Room | | Water | | | |
| 175 | 27 Mar. 2002 | Syrup | Tank JT 1 | Sanitation | 06 ufc | 03 ufc | — |
| | | Room | | Water | | | |
| 176 | 27 Mar. 2002 | Syrup | Tank JT 5 | Sanitation | 06 ufc | 02 ufc | — |
| | | Room | | Water | | | |
| 177 | 27 Mar. 2002 | Syrup | Dispenser | Sanitation | 02 ufc | 01 ufc | — |
| | | Room | Line | Water | | | |
| 178 | 27 Mar. 2002 | Unloading | Transport | Fructose | 00 ufc | 01 ufc | — |
| 179 | 27 Mar. 2002 | Unloading | Transport | Fructose | 02 ufc | 04 ufc | — |
| 180 | 27 Mar. 2002 | Unloading | Transport | Fructose | 03 ufc | 01 ufc | — |
| 181 | 27 Mar. 2002 | Unloading | Transport | Fructose | 00 ufc | 01 ufc | — |
| 182 | 27 Mar. 2002 | Unloading | Transport | Fructose | 01 ufc | 02 ufc | — |
| 183 | 27 Mar. 2002 | Unloading | Transport | Fructose | 00 ufc | 00 ufc | — |
| 184 | 27 Mar. 2002 | Syrup | Tank JT | Coca | 00 ufc | 01 ufc | — |
| | | Room | | Cola | | | |
| 185 | 27 Mar. 2002 | Syrup | Tank JT | Coca | 01 ufc | 00 ufc | — |
| | | Room | | Cola | | | |
| | | | | Light | | | |
| 186 | 27 Mar. 2002 | Syrup | Tank JT | Schweppes | 00 ufc | 00 ufc | — |
| | | Room | | Citrus | | | |
| 187 | 27 Mar. 2002 | Syrup | Tank JT | Coca | 02 ufc | 00 ufc | — |
| | | Room | | Cola | | | |
| 188 | 27 Mar. 2002 | Syrup | Tank JT | Coca | 00 ufc | 01 ufc | — |
| | | Room | | Cola | | | |
| 189 | 27 Mar. 2002 | Syrup | Tank JT | Coca | 02 ufc | 01 ufc | — |
| | | Room | | Cola | | | |
| | | | | Light | | | |
| 190 | 27 Mar. 2002 | Syrup | Tank JT | Schweppes | 01 ufc | 01 ufc | — |
| | | Room | | Ginger Ale | | | |
| 191 | 27 Mar. 2002 | Syrup | Tank JT | Coca | 03 ufc | 00 ufc | — |
| | | Room | | Cola | | | |
| | | | | Light | | | |
| 192 | 27 Mar. 2002 | Syrup | Tank JT | Coca | 00 ufc | 02 ufc | — |
| | | Room | | Cola | | | |
| 193 | 27 Mar. 2002 | Syrup | Tank JT | Tai Pink | 00 ufc | 00 ufc | — |
| | | Room | | Grapefruit | | | |
| 194 | 27 Mar. 2002 | Syrup | Tank JT | Coca | 00 ufc | 00 ufc | — |
| | | Room | | Cola | | | |
| | | | | Light | | | |
| 195 | 27 Mar. 2002 | Syrup | Tank JT | Coca | 00 ufc | 01 ufc | — |
| | | Room | | Cola | | | |
| 196 | 27 Mar. 2002 | Syrup | Tank JT | Coca | 01 ufc | 00 ufc | — |
| | | Room | | Cola | | | |
| 197 | 28 Mar. 2002 | Unloading | Transport | Fructose | 00 ufc | 01 ufc | — |
| 198 | 28 Mar. 2002 | Unloading | Transport | Fructose | 01 ufc | 00 ufc | — |
| 199 | 28 Mar. 2002 | Unloading | Transport | Fructose | 04 ufc | 01 ufc | — |
| 200 | 28 Mar. 2002 | Unloading | Transport | Fructose | 02 ufc | 00 ufc | — |
| 201 | 12 Apr. 2002 | Water | Outlet PC1 | Treated | 00 ufc | 02 ufc | — |
| | | Room | | Water | | | |
| 202 | 12 Apr. 2002 | Water | Outlet PC2 | Treated | 03 ufc | 04 ufc | — |
| | | Room | | Water | | | |
| 203 | 12 Apr. 2002 | Water | Outlet PC3 | Treated | 00 ufc | 04 ufc | — |
| | | Room | | Water | | | |
| 204 | 12 Apr. 2002 | Water | Outlet PC4 | Treated | 00 ufc | 01 ufc | — |
| | | Room | | Water | | | |
| 205 | 12 Apr. 2002 | Water | Outlet FP1 | Treated | 00 ufc | 00 ufc | — |
| | | Room | | Water | | | |
| 206 | 12 Apr. 2002 | Water | Outlet FP2 | Treated | 00 ufc | 00 ufc | — |
| | | Room | | Water | | | |
| 207 | 12 Apr. 2002 | Water | Outlet FP3 | Treated | 02 ufc | 00 ufc | — |
| | | Room | | Water | | | |
| 208 | 12 Apr. 2002 | Water | Outlet FP4 | Treated | 00 ufc | 00 ufc | — |
| | | Room | | Water | | | |
| 209 | 12 Apr. 2002 | Water | Tank 600 | Treated | 00 ufc | 00 ufc | — |
| | | Room | | Water | | | |
| 210 | 12 Apr. 2002 | Water | Tank 1050 | Raw | 00 ufc | 00 ufc | — |
| | | Room | | Water | | | |
| 211 | 12 Apr. 2002 | Water | Inlet FP1 | Treated | 00 ufc | 02 ufc | — |
| | | Room | | Water | | | |
| 212 | 12 Apr. 2002 | Water | Inlet FP2 | Treated | 00 ufc | 01 ufc | — |
| | | Room | | Water | | | |
| 213 | 16 Apr. 2002 | Quality | Laboratory | Prepared | 36 ufc | — | 44 ufc |
| | | Control | | | | | |
| 214 | 16 Apr. 2002 | Quality | Laboratory | Prepared | 42 ufc | — | 41 ufc |
| | | Control | | | | | |
| 215 | 16 Apr. 2002 | Quality | Laboratory | Prepared | 00 ufc | — | 00 ufc |
| | | Control | | | | | |
| 216 | 16 Apr. 2002 | Quality | Laboratory | Prepared | 01 ufc | — | 00 ufc |
| | | Control | | | | | |
| 217 | 16 Apr. 2002 | Quality | Laboratory | Prepared | 00 ufc | — | 03 ufc |
| | | Control | | | | | |
| 218 | 16 Apr. 2002 | Quality | Laboratory | Prepared | 00 ufc | — | 00 ufc |
| | | Control | | | | | |
| 219 | 16 Apr. 2002 | Quality | Laboratory | Prepared | 00 ufc | — | 00 ufc |
| | | Control | | | | | |
| 220 | 16 Apr. 2002 | Quality | Laboratory | Prepared | 00 ufc | — | 00 ufc |
| | | Control | | | | | |
| 221 | 16 Apr. 2002 | Quality | Laboratory | Prepared | 01 ufc | — | 01 ufc |
| | | Control | | | | | |
| 222 | 16 Apr. 2002 | Quality | Laboratory | Prepared | 00 ufc | — | 00 ufc |
| | | Control | | | | | |
| 223 | 16 Apr. 2002 | Quality | Laboratory | Prepared | 00 ufc | — | 00 ufc |
| | | Control | | | | | |
| 224 | 16 Apr. 2002 | Quality | Laboratory | Prepared | 00 ufc | — | 00 ufc |
| | | Control | | | | | |
| 225 | 23 Apr. 2002 | Quality | Laboratory | Prepared | 63 ufc | 58 ufc | — |
| | | Control | | | | | |
| 226 | 23 Apr. 2002 | Quality | Laboratory | Prepared | 52 ufc | 55 ufc | — |
| | | Control | | | | | |
| 227 | 23 Apr. 2002 | Quality | Laboratory | Prepared | 36 ufc | 30 ufc | — |
| | | Control | | | | | |
| 228 | 23 Apr. 2002 | Quality | Laboratory | Prepared | 15 ufc | 09 ufc | — |
| | | Control | | | | | |
| 229 | 23 Apr. 2002 | Quality | Laboratory | Prepared | 02 ufc | 00 ufc | — |
| | | Control | | | | | |
| 230 | 23 Apr. 2002 | Quality | Laboratory | Prepared | 08 ufc | 04 ufc | — |
| | | Control | | | | | |
| 231 | 23 Apr. 2002 | Quality | Laboratory | Prepared | 22 ufc | 17 ufc | — |
| | | Control | | | | | |
| 232 | 23 Apr. 2002 | Quality | Laboratory | Prepared | 00 ufc | 00 ufc | — |
| | | Control | | | | | |
| 233 | 23 Apr. 2002 | Quality | Laboratory | Prepared | 00 ufc | 00 ufc | — |
| | | Control | | | | | |
| 234 | 23 Apr. 2002 | Quality | Laboratory | Prepared | 00 ufc | 00 ufc | — |
| | | Control | | | | | |
| 235 | 23 Apr. 2002 | Quality | Laboratory | Prepared | 12 ufc | 10 ufc | — |
| | | Control | | | | | |
| 236 | 23 Apr. 2002 | Quality | Laboratory | Prepared | 126 ufc | 131 ufc | — |
| | | Control | | | | | |
| 237 | 23 Apr. 2002 | Quality | Laboratory | Prepared | 98 ufc | 106 ufc | — |
| | | Control | | | | | |
| 238 | 23 Apr. 2002 | Quality | Laboratory | Prepared | 82 ufc | 80 ufc | — |
| | | Control | | | | | |
| 239 | 23 Apr. 2002 | Quality | Laboratory | Prepared | 41 ufc | 35 ufc | — |
| | | Control | | | | | |
| 240 | 23 Apr. 2002 | Quality | Laboratory | Prepared | 37 ufc | 42 ufc | — |
| | | Control | | | | | |
| 241 | 23 Apr. 2002 | Quality | Laboratory | Prepared | 30 ufc | 35 ufc | — |
| | | Control | | | | | |
| 242 | 23 Apr. 2002 | Quality | Laboratory | Prepared | 22 ufc | 16 ufc | — |
| | | Control | | | | | |
| 243 | 23 Apr. 2002 | Quality | Laboratory | Prepared | 27 ufc | 20 ufc | — |
| | | Control | | | | | |
| 244 | 23 Apr. 2002 | Quality | Laboratory | Prepared | 29 ufc | 21 ufc | — |
| | | Control | | | | | |
| 245 | 23 Apr. 2002 | Quality | Laboratory | Prepared | 23 ufc | 27 ufc | — |
| | | Control | | | | | |
| 246 | 23 Apr. 2002 | Quality | Laboratory | Prepared | 00 ufc | 00 ufc | — |
| | | Control | | | | | |
| 247 | 23 Apr. 2002 | Quality | Laboratory | Prepared | 00 ufc | 00 ufc | — |
| | | Control | | | | | |
| 248 | 23 Apr. 2002 | Quality | Laboratory | Prepared | 02 ufc | 00 ufc | — |
| | | Control | | | | | |
| 249 | 23 Apr. 2002 | Quality | Laboratory | Prepared | 01 ufc | 00 ufc | — |
| | | Control | | | | | |
| 250 | 23 Apr. 2002 | Quality | Laboratory | Prepared | 00 ufc | 00 ufc | — |
| | | Control | | | | | |
| 251 | 3 May 2002 | Syrup | Gravimol 1 | Sanitation | 03 ufc | 01 ufc | — |
| | | Room | | Water | | | |
| 252 | 3 May 2002 | Syrup | Gravimol 1 | Sanitation | 00 ufc | 01 ufc | — |
| | | Room | | Water | | | |
| 253 | 3 May 2002 | Syrup | Gravimol 1 | Sanitation | 02 ufc | 01 ufc | — |
| | | Room | | Water | | | |
| 254 | 3 May 2002 | Syrup | Gravimol 2 | Sanitation | 02 ufc | 03 ufc | — |
| | | Room | | Water | | | |
| 255 | 3 May 2002 | Syrup | Gravimol 2 | Sanitation | 00 ufc | 00 ufc | — |
| | | Room | | Water | | | |
| 256 | 3 May 2002 | Syrup | Gravimol 2 | Sanitation | 02 ufc | 02 ufc | — |
| | | Room | | Water | | | |
| 257 | 3 May 2002 | Syrup | Gravimol 2 | Sanitation | 04 ufc | 02 ufc | — |
| | | Room | | Water | | | |
| 258 | 3 May 2002 | Syrup | Gravimol 2 | Sanitation | 04 ufc | 04 ufc | — |
| | | Room | | Water | | | |
| 259 | 3 May 2002 | Syrup | Tank JT 6 | Sanitation | 01 ufc | 01 ufc | — |
| | | Room | | Water | | | |
| 260 | 3 May 2002 | Syrup | Tank JT 1 | Sanitation | 03 ufc | 00 ufc | — |
| | | Room | | Water | | | |
| 261 | 3 May 2002 | Syrup | Tank JT 7 | Sanitation | 01 ufc | 03 ufc | — |
| | | Room | | Water | | | |
| 262 | 3 May 2002 | Syrup | Tank JT 8 | Sanitation | 00 ufc | 01 ufc | — |
| | | Room | | Water | | | |
| 263 | 3 May 2002 | Syrup | Tank JT 11 | Sanitation | 00 ufc | 01 ufc | — |
| | | Room | | Water | | | |
| 264 | 3 May 2002 | Syrup | Tank JT 3 | Sanitation | 01 ufc | 02 ufc | — |
| | | Room | | Water | | | |
| 265 | 3 May 2002 | Syrup | Tank JT 2 | Sanitation | 04 ufc | 01 ufc | — |
| | | Room | | Water | | | |
| 266 | 3 May 2002 | Syrup | Tank JT 7 | Sanitation | 02 ufc | 02 ufc | — |
| | | Room | | Water | | | |
| 267 | 3 May 2002 | Unloading | Transport | Fructose | 00 ufc | 00 ufc | — |
| 268 | 3 May 2002 | Unloading | Transport | Fructose | 02 ufc | 01 ufc | — |
| 269 | 3 May 2002 | Unloading | Transport | Fructose | 02 ufc | 00 ufc | — |
| 270 | 3 May 2002 | Unloading | Transport | Fructose | 00 ufc | 00 ufc | — |
| 271 | 3 May 2002 | Unloading | Transport | Fructose | 00 ufc | 00 ufc | — |
| 272 | 3 May 2002 | Unloading | Transport | Fructose | 01 ufc | 02 ufc | — |
| 273 | 3 May 2002 | Unloading | Transport | Fructose | 01 ufc | 00 ufc | — |
| 274 | 3 May 2002 | Unloading | Transport | Fructose | 00 ufc | 00 ufc | — |
| 275 | 3 May 2002 | Unloading | Transport | Fructose | 01 ufc | 02 ufc | — |
| 276 | 3 May 2002 | Unloading | Transport | Fructose | 05 ufc | 02 ufc | — |
| 277 | 3 May 2002 | Unloading | Transport | Fructose | 04 ufc | 02 ufc | — |
| 278 | 3 May 2002 | Unloading | Transport | Fructose | 00 ufc | 01 ufc | — |
| 279 | 3 May 2002 | Syrup | Tank JT | Fanta | 00 ufc | 00 ufc | — |
| | | Room | | Orange | | | |
| 280 | 3 May 2002 | Syrup | Tank JT | Tai Lime | 00 ufc | 00 ufc | — |
| | | Room | | Lemon | | | |
| 281 | 3 May 2002 | Syrup | Tank JT | Fanta | 00 ufc | 01 ufc | — |
| | | Room | | Tonic | | | |
| 282 | 3 May 2002 | Syrup | Tank JT | Coca | 01 ufc | 00 ufc | — |
| | | Room | | Cola | | | |
| | | | | Light | | | |
| 283 | 3 May 2002 | Syrup | Tank JT | Sprite | 00 ufc | 00 ufc | — |
| | | Room | | Light | | | |
| 284 | 3 May 2002 | Syrup | Tank JT | Sprite | 01 ufc | 00 ufc | — |
| | | Room | | | | | |
| 285 | 3 May 2002 | Syrup | Tank JT | Coca | 00 ufc | 00 ufc | — |
| | | Room | | Cola | | | |
| | | | | Light | | | |
| 286 | 3 May 2002 | Syrup | Tank JT | Coca | 02 ufc | 03 ufc | — |
| | | Room | | Cola | | | |
| 287 | 3 May 2002 | Syrup | Tank JT | Fanta | 00 ufc | 00 ufc | — |
| | | Room | | Orange | | | |
| 288 | 3 May 2002 | Syrup | Tank JT | Sprite | 00 ufc | 00 ufc | — |
| | | Room | | | | | |
| 289 | 3 May 2002 | Syrup | Tank JT | Coca | 03 ufc | 02 ufc | — |
| | | Room | | Cola | | | |
| 290 | 3 May 2002 | Syrup | Tank JT | Coca | 00 ufc | 00 ufc | — |
| | | Room | | Cola | | | |
| | | | | Light | | | |
| 291 | 3 May 2002 | Syrup | Tank JT | Quatro | 03 ufc | 03 ufc | — |
| | | Room | | Grapefruit | | | |
| 292 | 7 May 2002 | Quality | Laboratory | Prepared | 00 ufc | 00 ufc | — |
| | | Control | | | | | |
| 293 | 7 May 2002 | Quality | Laboratory | Prepared | 00 ufc | 00 ufc | — |
| | | Control | | | | | |
| 294 | 7 May 2002 | Quality | Laboratory | Prepared | 01 ufc | 00 ufc | — |
| | | Control | | | | | |
| 295 | 7 May 2002 | Quality | Laboratory | Prepared | 04 ufc | 02 ufc | — |
| | | Control | | | | | |
| 296 | 7 May 2002 | Quality | Laboratory | Prepared | 00 ufc | 00 ufc | — |
| | | Control | | | | | |
| 297 | 7 May 2002 | Quality | Laboratory | Prepared | 04 ufc | 04 ufc | — |
| | | Control | | | | | |
| 298 | 7 May 2002 | Quality | Laboratory | Prepared | 00 ufc | 01 ufc | — |
| | | Control | | | | | |
| 299 | 7 May 2002 | Quality | Laboratory | Prepared | 00 ufc | 00 ufc | — |
| | | Control | | | | | |
| 300 | 7 May 2002 | Quality | Laboratory | Prepared | 00 ufc | 00 ufc | — |
| | | Control | | | | | |
| 301 | 16 May 2002 | Quality | Laboratory | Prepared | 05 ufc | 03 ufc | — |
| | | Control | | | | | |
| 302 | 16 May 2002 | Quality | Laboratory | Prepared | 03 ufc | 00 ufc | — |
| | | Control | | | | | |
| 303 | 16 May 2002 | Quality | Laboratory | Prepared | 09 ufc | 06 ufc | — |
| | | Control | | | | | |
| 304 | 16 May 2002 | Quality | Laboratory | Prepared | 07 ufc | 05 ufc | — |
| | | Control | | | | | |
| 305 | 16 May 2002 | Quality | Laboratory | Prepared | 05 ufc | 08 ufc | — |
| | | Control | | | | | |
| 306 | 16 May 2002 | Quality | Laboratory | Prepared | 06 ufc | 03 ufc | — |
| | | Control | | | | | |
| 307 | 16 May 2002 | Quality | Laboratory | Prepared | 02 ufc | 00 ufc | — |
| | | Control | | | | | |
| 308 | 16 May 2002 | Quality | Laboratory | Prepared | 04 ufc | 01 ufc | — |
| | | Control | | | | | |
| 309 | 16 May 2002 | Quality | Laboratory | Prepared | 09 ufc | 07 ufc | — |
| | | Control | | | | | |
| 310 | 16 May 2002 | Quality | Laboratory | Prepared | 07 ufc | 02 ufc | — |
| | | Control | | | | | |
| 311 | 16 May 2002 | Quality | Laboratory | Prepared | 86 ufc | 79 ufc | — |
| | | Control | | | | | |
| 312 | 16 May 2002 | Quality | Laboratory | Prepared | 112 ufc | 104 ufc | — |
| | | Control | | | | | |
| 313 | 16 May 2002 | Quality | Laboratory | Prepared | 93 ufc | 108 ufc | — |
| | | Control | | | | | |
| 314 | 16 May 2002 | Quality | Laboratory | Prepared | 90 ufc | 99 ufc | — |
| | | Control | | | | | |
| 315 | 16 May 2002 | Quality | Laboratory | Prepared | 78 ufc | 81 ufc | — |
| | | Control | | | | | |
| 316 | 16 May 2002 | Quality | Laboratory | Prepared | 101 ufc | 89 ufc | — |
| | | Control | | | | | |
| 317 | 16 May 2002 | Quality | Laboratory | Prepared | 91 ufc | 82 ufc | — |
| | | Control | | | | | |
| 318 | 16 May 2002 | Quality | Laboratory | Prepared | 88 ufc | 90 ufc | — |
| | | Control | | | | | |
| 319 | 16 May 2002 | Quality | Laboratory | Prepared | 94 ufc | 90 ufc | — |
| | | Control | | | | | |
| 320 | 16 May 2002 | Quality | Laboratory | Prepared | 96 ufc | 84 ufc | — |
| | | Control | | | | | |
| 321 | 29 May 2001 | Bottling | Filling | Sanitation | 08 ufc | 05 ufc | — |
| | | | Valve | Water | | | |
| 322 | 29 May 2001 | Bottling | Filling | Sanitation | 10 ufc | 07 ufc | — |
| | | | Valve | Water | | | |
| 323 | 29 May 2001 | Bottling | Filling | Sanitation | 06 ufc | 05 ufc | — |
| | | | Valve | Water | | | |
| 324 | 29 May 2001 | Bottling | Filling | Sanitation | 05 ufc | 08 ufc | — |
| | | | Valve | Water | | | |
| 325 | 29 May 2001 | Bottling | Filling | Sanitation | 06 ufc | 03 ufc | — |
| | | | Valve | Water | | | |
| 326 | 29 May 2001 | Bottling | Filling | Sanitation | 05 ufc | 07 ufc | — |
| | | | Valve | Water | | | |
| 327 | 29 May 2001 | Bottling | Filling | Sanitation | 07 ufc | 07 ufc | — |
| | | | Valve | Water | | | |
| 328 | 29 May 2001 | Bottling | Filling | Sanitation | 09 ufc | 06 ufc | — |
| | | | Valve | Water | | | |
| 329 | 29 May 2001 | Bottling | Filling | Sanitation | 02 ufc | 00 ufc | — |
| | | | Valve | Water | | | |
| 330 | 29 May 2001 | Bottling | Filling | Sanitation | 04 ufc | 06 ufc | — |
| | | | Valve | Water | | | |
| 331 | 29 May 2001 | Bottling | Filling | Sanitation | 05 ufc | 02 ufc | — |
| | | | Valve | Water | | | |
| 332 | 29 May 2001 | Bottling | Filling | Sanitation | 08 ufc | 08 ufc | — |
| | | | Valve | Water | | | |
| 333 | 14 Jun. 2001 | Quality | Laboratory | Prepared | 00 ufc | 00 ufc | 00 ufc |
| | | Control | | | | | |
| 334 | 14 Jun. 2001 | Quality | Laboratory | Prepared | 15 ufc | 09 ufc | 09 ufc |
| | | Control | | | | | |
| 335 | 14 Jun. 2001 | Quality | Laboratory | Prepared | 15 ufc | 18 ufc | 16 ufc |
| | | Control | | | | | |
| 336 | 14 Jun. 2001 | Quality | Laboratory | Prepared | 01 ufc | 01 ufc | 00 ufc |
| | | Control | | | | | |
| 337 | 14 Jun. 2001 | Quality | Laboratory | Prepared | 00 ufc | 00 ufc | 00 ufc |
| | | Control | | | | | |
| 338 | 14 Jun. 2001 | Quality | Laboratory | Prepared | 00 ufc | 00 ufc | 00 ufc |
| | | Control | | | | | |
| 339 | 3 Jul. 2001 | Quality | Laboratory | Prepared | TNTC | TNTC | — |
| | | Control | | | | | |
| 340 | 3 Jul. 2001 | Quality | Laboratory | Prepared | TNTC | TNTC | — |
| | | Control | | | | | |
| 341 | 3 Jul. 2001 | Quality | Laboratory | Prepared | TNTC | TNTC | — |
| | | Control | | | | | |
| 342 | 3 Jul. 2001 | Quality | Laboratory | Prepared | 01 ufc | 00 ufc | — |
| | | Control | | | | | |
| 343 | 3 Jul. 2001 | Quality | Laboratory | Prepared | 00 ufc | 00 ufc | — |
| | | Control | | | | | |
| 344 | 3 Jul. 2001 | Quality | Laboratory | Prepared | TNTC | TNTC | — |
| | | Control | | | | | |
| 345 | 24 Jul. 2002 | Line 2 | Filling | Sanitation | 01 ufc | 00 ufc | — |
| | | | Valve | Water | | | |
| 346 | 24 Jul. 2002 | Line 2 | Filling | Sanitation | 08 ufc | 05 ufc | — |
| | | | Valve | Water | | | |
| 347 | 24 Jul. 2002 | Line 2 | Filling | Sanitation | 03 ufc | 02 ufc | — |
| | | | Valve | Water | | | |
| 348 | 24 Jul. 2002 | Line 2 | Filling | Sanitation | 00 ufc | 01 ufc | — |
| | | | Valve | Water | | | |
| 349 | 25 Jul. 2002 | Line 2 | Filling | Sanitation | 02 ufc | 03 ufc | — |
| | | | Valve | Water | | | |
| 350 | 25 Jul. 2002 | Line 2 | Filling | Sanitation | 02 ufc | 04 ufc | — |
| | | | Valve | Water | | | |
| 351 | 25 Jul. 2002 | Line 2 | Filling | Sanitation | 04 ufc | 01 ufc | — |
| | | | Valve | Water | | | |
| 52 | 25 Jul. 2002 | Line 2 | Filling | Sanitation | 02 ufc | 03 ufc | — |
| | | | Valve | Water | | | |
| 353 | 25 Jul. 2002 | Line 2 | Filling | Sanitation | 06 ufc | 08 ufc | — |
| | | | Valve | Water | | | |
| 354 | 25 Jul. 2002 | Line 2 | Filling | Sanitation | 07 ufc | 10 ufc | — |
| | | | Valve | Water | | | |
| 355 | 25 Jul. 2002 | Line 2 | Filling | Sanitation | 09 ufc | 07 ufc | — |
| | | | Valve | Water | | | |
| 356 | 25 Jul. 2002 | Line 2 | Filling | Sanitation | 05 ufc | 07 ufc | — |
| | | | Valve | Water | | | |
| 357 | 25 Jul. 2002 | Line 2 | Filling | Sanitation | 10 ufc | 08 ufc | — |
| | | | Valve | Water | | | |
| 358 | 25 Jul. 2002 | Line 2 | Filling | Sanitation | 06 ufc | 08 ufc | — |
| | | | Valve | Water | | | |
| 359 | 25 Jul. 2002 | Line 2 | Filling | Sanitation | 04 ufc | 07 ufc | — |
| | | | Valve | Water | | | |
| 360 | 25 Jul. 2002 | Line 2 | Filling | Sanitation | 08 ufc | 08 ufc | — |
| | | | Valve | Water | | | |
| 361 | 26 Jul. 2002 | Line 2 | Filling | Sanitation | 04 ufc | 06 ufc | — |
| | | | Valve | Water | | | |
| 362 | 26 Jul. 2002 | Line 2 | Filling | Sanitation | 08 ufc | 05 ufc | — |
| | | | Valve | Water | | | |
| 363 | 26 Jul. 2002 | Line 2 | Filling | Sanitation | 10 ufc | 07 ufc | — |
| | | | Valve | Water | | | |
| 364 | 26 Jul. 2002 | Line 2 | Filling | Sanitation | 09 ufc | 07 ufc | — |
| | | | Valve | Water | | | |
| 365 | 27 Jul. 2002 | Line 2 | Filling | Sanitation | 05 ufc | 06 ufc | — |
| | | | Valve | Water | | | |
| 366 | 27 Jul. 2002 | Line 2 | Filling | Sanitation | 07 ufc | 06 ufc | — |
| | | | Valve | Water | | | |
| 367 | 27 Jul. 2002 | Line 2 | Filling | Sanitation | 05 ufc | 02 ufc | — |
| | | | Valve | Water | | | |
| 368 | 27 Jul. 2002 | Line 2 | Filling | Sanitation | 05 ufc | 06 ufc | — |
| | | | Valve | Water | | | |
| 369 | 29 Jul. 2002 | Line 2 | Filling | Sanitation | 04 ufc | 02 ufc | — |
| | | | Valve | Water | | | |
| 370 | 29 Jul. 2002 | Line 2 | Filling | Sanitation | 02 ufc | 03 ufc | — |
| | | | Valve | Water | | | |
| 371 | 29 Jul. 2002 | Line 2 | Filling | Sanitation | 01 ufc | 00 ufc | — |
| | | | Valve | Water | | | |
| 372 | 29 Jul. 2002 | Line 2 | Filling | Sanitation | 03 ufc | 05 ufc | — |
| | | | Valve | Water | | | |
| 373 | 29 Jul. 2002 | Line 2 | Filling | Sanitation | 04 ufc | 08 ufc | — |
| | | | Valve | Water | | | |
| 374 | 29 Jul. 2002 | Line 2 | Filling | Sanitation | 10 ufc | 09 ufc | — |
| | | | Valve | Water | | | |
| 375 | 29 Jul. 2002 | Line 2 | Filling | Sanitation | 06 ufc | 07 ufc | — |
| | | | Valve | Water | | | |
| 376 | 29 Jul. 2002 | Line 2 | Filling | Sanitation | 06 ufc | 08 ufc | — |
| | | | Valve | Water | | | |
| 377 | 30 Jul. 2002 | Line 2 | Filling | Sanitation | 07 ufc | 09 ufc | — |
| | | | Valve | Water | | | |
| 378 | 30 Jul. 2002 | Line 2 | Filling | Sanitation | 10 ufc | 08 ufc | — |
| | | | Valve | Water | | | |
| 379 | 30 Jul. 2002 | Line 2 | Filling | Sanitation | 09 ufc | 06 ufc | — |
| | | | Valve | Water | | | |
| 380 | 30 Jul. 2002 | Line 2 | Filling | Sanitation | 07 ufc | 08 ufc | — |
| | | | Valve | Water | | | |
| 381 | 30 Jul. 2002 | Line 2 | Filling | Sanitation | 05 ufc | 09 ufc | — |
| | | | Valve | Water | | | |
| 382 | 30 Jul. 2002 | Line 2 | Filling | Sanitation | 08 ufc | 09 ufc | — |
| | | | Valve | Water | | | |
| 383 | 30 Jul. 2002 | Line 2 | Filling | Sanitation | 08 ufc | 08 ufc | — |
| | | | Valve | Water | | | |
| 384 | 30 Jul. 2002 | Line 2 | Filling | Sanitation | 09 ufc | 06 ufc | — |
| | | | Valve | Water | | | |
| 385 | 30 Jul. 2002 | Line 2 | Filling | Sanitation | 09 ufc | 08 ufc | — |
| | | | Valve | Water | | | |
| 386 | 30 Jul. 2002 | Line 2 | Filling | Sanitation | 06 ufc | 08 ufc | — |
| | | | Valve | Water | | | |
| 387 | 30 Jul. 2002 | Line 2 | Filling | Sanitation | 08 ufc | 06 ufc | — |
| | | | Valve | Water | | | |
| 388 | 30 Jul. 2002 | Line 2 | Filling | Sanitation | 07 ufc | 05 ufc | — |
| | | | Valve | Water | | | |
| 389 | 31 Jul. 2002 | Line 2 | Filling | Sanitation | 09 ufc | 09 ufc | — |
| | | | Valve | Water | | | |
| 390 | 31 Jul. 2002 | Line 2 | Filling | Sanitation | 09 ufc | 08 ufc | — |
| | | | Valve | Water | | | |
| 391 | 31 Jul. 2002 | Line 2 | Filling | Sanitation | 04 ufc | 07 ufc | — |
| | | | Valve | Water | | | |
| 392 | 31 Jul. 2002 | Line 2 | Filling | Sanitation | 07 ufc | 09 ufc | — |
| | | | Valve | Water | | | |
| 393 | 1 Aug. 2002 | Line 2 | Filling | Sanitation | 05 ufc | 03 ufc | — |
| | | | Valve | Water | | | |
| 394 | 1 Aug. 2002 | Line 2 | Filling | Sanitation | 06 ufc | 06 ufc | — |
| | | | Valve | Water | | | |
| 395 | 1 Aug. 2002 | Line 2 | Filling | Sanitation | 06 ufc | 05 ufc | — |
| | | | Valve | Water | | | |
| 396 | 1 Aug. 2002 | Line 2 | Filling | Sanitation | 08 ufc | 05 ufc | — |
| | | | Valve | Water | | | |
| 397 | 2 Aug. 2002 | Line 2 | Filling | Sanitation | 09 ufc | 07 ufc | — |
| | | | Valve | Water | | | |
| 398 | 2 Aug. 2002 | Line 2 | Filling | Sanitation | 06 ufc | 06 ufc | — |
| | | | Valve | Water | | | |
| 399 | 2 Aug. 2002 | Line 2 | Filling | Sanitation | 06 ufc | 08 ufc | — |
| | | | Valve | Water | | | |
| 400 | 2 Aug. 2002 | Line 2 | Filling | Sanitation | 07 ufc | 05 ufc | — |
| | | | Valve | Water | | | |
| 401 | 2 Aug. 2002 | Line 2 | Filling | Sanitation | 02 ufc | 04 ufc | — |
| | | | Valve | Water | | | |
| 402 | 2 Aug. 2002 | Line 2 | Filling | Sanitation | 05 ufc | 05 ufc | — |
| | | | Valve | Water | | | |
| 403 | 2 Aug. 2002 | Line 2 | Filling | Sanitation | 05 ufc | 02 ufc | — |
| | | | Valve | Water | | | |
| 404 | 2 Aug. 2002 | Line 2 | Filling | Sanitation | 09 ufc | 05 ufc | — |
| | | | Valve | Water | | | |
| 405 | 3 Aug. 2002 | Line 2 | Filling | Sanitation | 06 ufc | 03 ufc | — |
| | | | Valve | Water | | | |
| 406 | 3 Aug. 2002 | Line 2 | Filling | Sanitation | 08 ufc | 05 ufc | — |
| | | | Valve | Water | | | |
| 407 | 3 Aug. 2002 | Line 2 | Filling | Sanitation | 00 ufc | 02 ufc | — |
| | | | Valve | Water | | | |
| 408 | 3 Aug. 2002 | Line 2 | Filling | Sanitation | 04 ufc | 02 ufc | — |
| | | | Valve | Water | | | |
| 409 | 3 Aug. 2002 | Line 2 | Filling | Sanitation | 03 ufc | 07 ufc | — |
| | | | Valve | Water | | | |
| 410 | 3 Aug. 2002 | Line 2 | Filling | Sanitation | 10 ufc | 10 ufc | — |
| | | | Valve | Water | | | |
| 411 | 3 Aug. 2002 | Line 2 | Filling | Sanitation | 07 ufc | 09 ufc | — |
| | | | Valve | Water | | | |
| 412 | 3 Aug. 2002 | Line 2 | Filling | Sanitation | 09 ufc | 09 ufc | — |
| | | | Valve | Water | | | |
| 413 | 5 Aug. 2002 | Line 2 | Filling | Sanitation | 06 ufc | 05 ufc | — |
| | | | Valve | Water | | | |
| 414 | 5 Aug. 2002 | Line 2 | Filling | Sanitation | 05 ufc | 07 ufc | — |
| | | | Valve | Water | | | |
| 415 | 5 Aug. 2002 | Line 2 | Filling | Sanitation | 03 ufc | 06 ufc | — |
| | | | Valve | Water | | | |
| 416 | 5 Aug. 2002 | Line 2 | Filling | Sanitation | 04 ufc | 06 ufc | — |
| | | | Valve | Water | | | |
| 417 | 7 Aug. 2002 | Line 2 | Filling | Sanitation | 05 ufc | 02 ufc | — |
| | | | Valve | Water | | | |
| 418 | 7 Aug. 2002 | Line 2 | Filling | Sanitation | 06 ufc | 02 ufc | — |
| | | | Valve | Water | | | |
| 419 | 7 Aug. 2002 | Line 2 | Filling | Sanitation | 08 ufc | 06 ufc | — |
| | | | Valve | Water | | | |
| 420 | 7 Aug. 2002 | Line 2 | Filling | Sanitation | 09 ufc | 09 ufc | — |
| | | | Valve | Water | | | |
| 421 | 8 Aug. 2002 | Line 2 | Filling | Sanitation | 02 ufc | 05 ufc | — |
| | | | Valve | Water | | | |
| 422 | 8 Aug. 2002 | Line 2 | Filling | Sanitation | 08 ufc | 07 ufc | — |
| | | | Valve | Water | | | |
| 423 | 8 Aug. 2002 | Line 2 | Filling | Sanitation | 07 ufc | 02 ufc | — |
| | | | Valve | Water | | | |
| 424 | 8 Aug. 2002 | Line 2 | Filling | Sanitation | 10 ufc | 06 ufc | — |
| | | | Valve | Water | | | |
| 425 | 8 Aug. 2002 | Line 2 | Filling | Sanitation | 09 ufc | 04 ufc | — |
| | | | Valve | Water | | | |
| 426 | 8 Aug. 2002 | Line 2 | Filling | Sanitation | 05 ufc | 07 ufc | — |
| | | | Valve | Water | | | |
| 427 | 9 Aug. 2002 | Line 2 | Filling | Sanitation | 07 ufc | 07 ufc | — |
| | | | Valve | Water | | | |
| 428 | 9 Aug. 2002 | Line 2 | Filling | Sanitation | 04 ufc | 09 ufc | — |
| | | | Valve | Water | | | |
| 429 | 9 Aug. 2002 | Line 2 | Filling | Sanitation | 05 ufc | 03 ufc | — |
| | | | Valve | Water | | | |
| 430 | 9 Aug. 2002 | Line 2 | Filling | Sanitation | 07 ufc | 07 ufc | — |
| | | | Valve | Water | | | |
| 431 | 10 Aug. 2002 | Line 2 | Filling | Sanitation | 06 ufc | 07 ufc | — |
| | | | Valve | Water | | | |
| 432 | 10 Aug. 2002 | Line 2 | Filling | Sanitation | 08 ufc | 08 ufc | — |
| | | | Valve | Water | | | |
| 433 | 10 Aug. 2002 | Line 2 | Filling | Sanitation | 09 ufc | 08 ufc | — |
| | | | Valve | Water | | | |
| 434 | 10 Aug. 2002 | Line 2 | Filling | Sanitation | 06 ufc | 07 ufc | — |
| | | | Valve | Water | | | |
| 435 | 10 Aug. 2002 | Line 2 | Filling | Sanitation | 04 ufc | 04 ufc | — |
| | | | Valve | Water | | | |
| 436 | 10 Aug. 2002 | Line 2 | Filling | Sanitation | 03 ufc | 02 ufc | — |
| | | | Valve | Water | | | |
| 437 | 10 Aug. 2002 | Line 2 | Filling | Sanitation | 00 ufc | 00 ufc | — |
| | | | Valve | Water | | | |
| 438 | 10 Aug. 2002 | Line 2 | Filling | Sanitation | 00 ufc | 00 ufc | — |
| | | | Valve | Water | | | |
| 439 | 12 Aug. 2002 | Line 2 | Filling | Sanitation | 05 ufc | 05 ufc | — |
| | | | Valve | Water | | | |
| 440 | 12 Aug. 2002 | Line 2 | Filling | Sanitation | 06 ufc | 05 ufc | — |
| | | | Valve | Water | | | |
| 441 | 12 Aug. 2002 | Line 2 | Filling | Sanitation | 02 ufc | 03 ufc | — |
| | | | Valve | Water | | | |
| 442 | 12 Aug. 2002 | Line 2 | Filling | Sanitation | 05 ufc | 07 ufc | — |
| | | | Valve | Water | | | |
| 443 | 12 Aug. 2002 | Line 2 | Filling | Sanitation | 04 ufc | 07 ufc | — |
| | | | Valve | Water | | | |
| 444 | 12 Aug. 2002 | Line 2 | Filling | Sanitation | 08 ufc | 09 ufc | — |
| | | | Valve | Water | | | |
| 445 | 12 Aug. 2002 | Line 2 | Filling | Sanitation | 10 ufc | 08 ufc | — |
| | | | Valve | Water | | | |
| 446 | 12 Aug. 2002 | Line 2 | Filling | Sanitation | 05 ufc | 06 ufc | — |
| | | | Valve | Water | | | |
| 447 | 13 Aug. 2002 | Line 2 | Filling | Sanitation | 05 ufc | 05 ufc | — |
| | | | Valve | Water | | | |
| 448 | 13 Aug. 2002 | Line 2 | Filling | Sanitation | 06 ufc | 04 ufc | — |
| | | | Valve | Water | | | |
| 449 | 13 Aug. 2002 | Line 2 | Filling | Sanitation | 08 ufc | 04 ufc | — |
| | | | Valve | Water | | | |
| 450 | 13 Aug. 2002 | Line 2 | Filling | Sanitation | 05 ufc | 04 ufc | — |
| | | | Valve | Water | | | |
| 451 | 13 Aug. 2002 | Line 2 | Filling | Sanitation | 05 ufc | 04 ufc | — |
| | | | Valve | Water | | | |
| 452 | 13 Aug. 2002 | Line 2 | Filling | Sanitation | 04 ufc | 02 ufc | — |
| | | | Valve | Water | | | |
| 453 | 13 Aug. 2002 | Line 2 | Filling | Sanitation | 01 ufc | 00 ufc | — |
| | | | Valve | Water | | | |
| 454 | 13 Aug. 2002 | Line 2 | Filling | Sanitation | 00 ufc | 00 ufc | — |
| | | | Valve | Water | | | |
| 455 | 14 Aug. 2002 | Line 2 | Filling | Sanitation | 00 ufc | 00 ufc | — |
| | | | Valve | Water | | | |
| 456 | 14 Aug. 2002 | Line 2 | Filling | Sanitation | 02 ufc | 00 ufc | — |
| | | | Valve | Water | | | |
| 457 | 14 Aug. 2002 | Line 2 | Filling | Sanitation | 02 ufc | 02 ufc | — |
| | | | Valve | Water | | | |
| 458 | 14 Aug. 2002 | Line 2 | Filling | Sanitation | 01 ufc | 04 ufc | — |
| | | | Valve | Water | | | |
| 459 | 14 Aug. 2002 | Line 2 | Filling | Sanitation | 00 ufc | 02 ufc | — |
| | | | Valve | Water | | | |
| 460 | 14 Aug. 2002 | Line 2 | Filling | Sanitation | 04 ufc | 03 ufc | — |
| | | | Valve | Water | | | |
| 461 | 14 Aug. 2002 | Line 2 | Filling | Sanitation | 01 ufc | 00 ufc | — |
| | | | Valve | Water | | | |
| 462 | 14 Aug. 2002 | Line 2 | Filling | Sanitation | 02 ufc | 02 ufc | — |
| | | | Valve | Water | | | |
| 463 | 14 Aug. 2002 | Line 2 | Filling | Sanitation | 02 ufc | 01 ufc | — |
| | | | Valve | Water | | | |
| 464 | 14 Aug. 2002 | Line 2 | Filling | Sanitation | 00 ufc | 01 ufc | — |
| | | | Valve | Water | | | |
| 465 | 14 Aug. 2002 | Line 2 | Filling | Sanitation | 00 ufc | 00 ufc | — |
| | | | Valve | Water | | | |
| 466 | 14 Aug. 2002 | Line 2 | Filling | Sanitation | 01 ufc | 02 ufc | — |
| | | | Valve | Water | | | |
| 467 | 15 Aug. 2002 | Line 2 | Filling | Sanitation | 04 ufc | 03 ufc | — |
| | | | Valve | Water | | | |
| 468 | 15 Aug. 2002 | Line 2 | Filling | Sanitation | 05 ufc | 08 ufc | — |
| | | | Valve | Water | | | |
| 469 | 15 Aug. 2002 | Line 2 | Filling | Sanitation | 09 ufc | 08 ufc | — |
| | | | Valve | Water | | | |
| 470 | 15 Aug. 2002 | Line 2 | Filling | Sanitation | 04 ufc | 03 ufc | — |
| | | | Valve | Water | | | |
| 471 | 15 Aug. 2002 | Line 2 | Filling | Sanitation | 05 ufc | 04 ufc | — |
| | | | Valve | Water | | | |
| 472 | 15 Aug. 2002 | Line 2 | Filling | Sanitation | 05 ufc | 05 ufc | — |
| | | | Valve | Water | | | |
| 473 | 15 Aug. 2002 | Line 2 | Filling | Sanitation | 03 ufc | 05 ufc | — |
| | | | Valve | Water | | | |
| 474 | 15 Aug. 2002 | Line 2 | Filling | Sanitation | 03 ufc | 03 ufc | — |
| | | | Valve | Water | | | |
| 475 | 16 Aug. 2002 | Line 2 | Filling | Sanitation | 04 ufc | 06 ufc | — |
| | | | Valve | Water | | | |
| 476 | 16 Aug. 2002 | Line 2 | Filling | Sanitation | 05 ufc | 05 ufc | — |
| | | | Valve | Water | | | |
| 477 | 16 Aug. 2002 | Line 2 | Filling | Sanitation | 05 ufc | 07 ufc | — |
| | | | Valve | Water | | | |
| 478 | 16 Aug. 2002 | Line 2 | Filling | Sanitation | 09 ufc | 07 ufc | — |
| | | | Valve | Water | | | |
| 479 | 17 Aug. 2002 | Line 2 | Syrup | Sanitation | 08 ufc | 05 ufc | — |
| | | | Tank | Water | | | |
| 480 | 17 Aug. 2002 | Line 2 | Beverage | Sanitation | 07 ufc | 07 ufc | — |
| | | | Tank | Water | | | |
| 481 | 6 Sep. 2002 | Syrup | Tank JT 4 | Sanitation | 00 ufc | 00 ufc | — |
| | | Room | | Water | | | |
| 482 | 6 Sep. 2002 | Syrup | Tank JT 3 | Sanitation | 01 ufc | 00 ufc | — |
| | | Room | | Water | | | |
| 483 | 6 Sep. 2002 | Syrup | Tank JT 2 | Sanitation | 01 ufc | 01 ufc | — |
| | | Room | | Water | | | |
| 484 | 6 Sep. 2002 | Syrup | Tank JT 1 | Sanitation | 01 ufc | 00 ufc | — |
| | | Room | | Water | | | |
| 485 | 6 Sep. 2002 | Syrup | Tank JT 5 | Sanitation | 00 ufc | 00 ufc | — |
| | | Room | | Water | | | |
| 486 | 6 Sep. 2002 | Syrup | Tank JT 1 | Sanitation | 00 ufc | 00 ufc | — |
| | | Room | | Water | | | |
| 487 | 6 Sep. 2002 | Syrup | Tank JT 11 | Sanitation | 00 ufc | 02 ufc | — |
| | | Room | | Water | | | |
| 488 | 7 Sep. 2002 | Syrup | Tank JT 6 | Sanitation | 02 ufc | 01 ufc | — |
| | | Room | | Water | | | |
| 489 | 7 Sep. 2002 | Syrup | Tank JT 9 | Sanitation | 00 ufc | 00 ufc | — |
| | | Room | | Water | | | |
| 490 | 7 Sep. 2002 | Syrup | Tank JT 4 | Sanitation | 00 ufc | 00 ufc | — |
| | | Room | | Water | | | |
| 491 | 7 Sep. 2002 | Syrup | Tank JT 3 | Sanitation | 01 ufc | 02 ufc | — |
| | | Room | | Water | | | |
| 492 | 7 Sep. 2002 | Syrup | Tank JT 1 | Sanitation | 00 ufc | 00 ufc | — |
| | | Room | | Water | | | |
| 493 | 7 Sep. 2002 | Syrup | Tank JT 11 | Sanitation | 00 ufc | 01 ufc | — |
| | | Room | | Water | | | |
| 494 | 9 Sep. 2002 | Syrup | Tank JT 5 | Sanitation | 00 ufc | 00 ufc | — |
| | | Room | | Water | | | |
| 495 | 9 Sep. 2002 | Syrup | Tank JT 10 | Sanitation | 00 ufc | 00 ufc | — |
| | | Room | | Water | | | |
| 496 | 9 Sep. 2002 | Syrup | Tank JT 3 | Sanitation | 01 ufc | 00 ufc | — |
| | | Room | | Water | | | |
| 497 | 9 Sep. 2002 | Syrup | Tank JT 1 | Sanitation | 00 ufc | 01 ufc | — |
| | | Room | | Water | | | |
| 498 | 10 Sep. 2002 | Syrup | Tank JT 4 | Sanitation | 00 ufc | 00 ufc | — |
| | | Room | | Water | | | |
| 499 | 10 Sep. 2002 | Syrup | Tank JT 5 | Sanitation | 03 ufc | 01 ufc | — |
| | | Room | | Water | | | |
| 500 | 10 Sep. 2002 | Syrup | Tank JT 6 | Sanitation | 01 ufc | 00 ufc | — |
| | | Room | | Water | | | |

The underlined tests in the above Table 4 correspond to inefficiencies in the preparation of the three culture mediums.

The culture medium composition of the invention keeps its characteristics and effectiveness after 6 months at 4^{a}C and therefore it may be considered that the inventive composition has a long life, thus allowing to work with high production indexes.

Conclusions:

1. The culture medium composition of the invention allows the production costs of culture mediums used for quality controls to decrease because of the low costs of its components as compared to other commercial culture mediums.

2. The components of the medium rapidly dissolve in an aqueous medium and the obtained composition does not require a filtration step for eliminating impurities.

3. The pH is easily adjusted.

4. The preparation of the inventive composition takes a short time.

5. The colonies grown in the composition are small thus making the counting thereof easier.

6. The composition may be prepared in large volumes and it is easily sterilized by techniques like autoclave, filtration.

7. The composition has an average life of one year at 4^{a}C.

This invention is better illustrated in the following examples that should not be construed as restrictive to the scope of the invention. On the contrary, it should be clearly understood that, after reading the present disclosure, other embodiments, modifications and equivalents of the invention will be apparent to those persons skilled in the art without departing from the spirit of the invention and/or the scope of the appended claims.

### EXAMPLE 1

Method of Elaborating the Culture Medium Composition of the Invention.

5.0 g of monobasic potassium phosphate, 1.0 of ammonium chloride, 1.0 g of heptahydrate magnesium sulfate and 40.0 g of D(+) saccharose have been mixed in a precipitate glass having a capacity for 1000 ml of final solution. Ultra pure water has been added up to 1 litre volume. The solution has been stirred or agitated by a magnetic stirrer up to having the components dissolved.

While maintaining a constant stirring a pre-calibrated and normalized electrode was introduced into the solution. The pH of the medium was measured and adjusted to a final 5 value by dripping a 1N sodium hydroxide solution.

The resulting solution was poured into an autoclave resistant flask and the flask was properly sealed. The medium was sterilized in autoclave at a temperature of 121° C. for 15 min.

After the sterilization in autoclave the medium was conserved refrigerated at a temperature of about 4° C.

### EXAMPLE 2

Comparative Analysis of the Efficiency of the Inventive Composition and Conventional Culture Mediums.

Fungi and yeast colonies have been dispersed in 500 ml ultra pure water and the following dilutions have been made 1/10², 1/10⁴, 1/10⁶, 1/10⁸, 1/10¹⁰, 1/10¹², 1/10¹⁴, 1/10¹⁶, 1/10¹⁸, 1/10²⁰.

In addition 12 Petri plates have been prepared for each culture medium. Each plate contained 2.5 ml of culture medium placed into the culture plate that had an absorbent surface “pad”.

20 ml of each sample were spread into each plate and incubated at 28° C. for 72 hours.

Comparative analysis were then carried out of samples taken from the sanitation water from the filling valves, as it is explained below.

Analysis of the sanitation samples:

After finishing the sanitation of the production line bacteriologic samples have been drawn of the final sanitation rinsing water. The samples have been taken from the filling valves after blazing thereof and the samples were placed into 120 ml sterile flasks. Then the samples were filtrated through a membrane with 0.8 micron pores, under entire aseptic conditions. The filters were removed and placed into Petri plates with pads and containing 3 ml of corresponding culture medium. Each plate was maintained for 72 hours at 28° C. and the colonies were then counted.

Comparative analysis of fungi-contaminated samples were also carried out with such samples having different contamination levels or microorganisms contents.

To that purpose samples of water intentionally contaminated with fungi at high concentrations (higher than 400 ufc), half concentrations (close to 100 ufc) and low concentrations (below 10 ufc) have been prepared. The samples were filtrated through a membrane with 0.8 micron pores. The filters were removed and placed into Petri plates with pads and containing 3 ml of each culture medium. The plates were maintained at 28° C. for 72 hours. After the incubation period the plates were removed and the colonies were counted.

### EXAMPLE 3

Study of the Life of the Inventive Composition.

A flask containing 1000 ml of the inventive culture composition was placed in a room at 4° C., with the room properly sterilized.

Time tests of the life of the inventive composition were simultaneously carried out. To this purpose the culture medium was fractioned into 2.5 ml vials that were sterilized and conserved at 4° C. The vials were removed one by one for 6 months and their effectiveness for culturing fungi and yeast were tested. The test in Petri plates was the same disclosed in the prior Example. Samples intentionally contaminated with fungi and yeast were employed as positive controls, and membranes through which the sterile culture medium was passed were employed as negative controls.

While preferred embodiments of the present invention have been illustrated and described, it will be obvious to those skilled in the art that various changes and modifications may be made therein without departing from the scope of the invention as defined in the appended claims.

We claim:

1. A culture medium comprising:
i. between about 4.5 g/l and about 5.5 g/l of monobasic potassium phosphate;
ii. between about 0.5 g/l and about 1.5 g/l of ammonium chloride;
iii. between about 0.5 g/l and about 1.5 g/l of heptahydrate magnesium sulfate;
iv. between about 30.0 g/l and about 50.0 g/l of D(+) saccharose, and
v. water.

2. The medium of claim 1, wherein the medium is for the culture of fungi and yeast.

3. The medium of claim 2, wherein the fungi and yeast are selected from the group consisting of Basillus Subtilis, Candida Albicans, Saccharomyces Cerevisiae, Saccharomyces Uvarum, Rhodotorula Rubra, Penicillium Camemberti, Aspergillus Niger, Trychophyton Ajelloi and Geotrichum Candidum.

4. The medium of claim 1, wherein the pH of the medium is between 4.5 and 5.5.

5. A method of preparing a culture medium comprising:
i. between about 4.5 g/l and about 5.5 g/l of monobasic potassium phosphate;
ii. between about 0.5 g/l and about 1.5 g/l of ammonium chloride;
iii. between about 0.5 g/l and about 1.5 g/l of heptahydrate magnesium sulfate;
iv. between about 30.0 g/l and about 50.0 g/l of D(+) saccharose, and
v. water, wherein the method comprises the steps of:
a. obtaining a solution by dissolving in ultra pure water, under agitation, the monobasic potassium phosphate, the ammonium chloride, the heptahydrate magnesium sulfate and the saccharose;
b. adjusting the pH of the solution obtained in step a. to 5.0, and
c. sterilizing the solution and conserving the solution at a temperature of 4^{a}C.

6. A culture medium composition for detecting fungi and/or yeast in installations, the composition comprising monobasic potassium phosphate, ammonium chloride, heptahydrate magnesium sulfate, D(+) saccharose, and water.

7. The composition of claim 6, wherein the composition comprises:
i. between about 4.5 g/l and about 5.5 g/l of monobasic potassium phosphate;
ii. between about 0.5 g/l and about 1.5 g/l of ammonium chloride;
iii. between about 0.5 g/l and about 1.5 g/l of heptahydrate magnesium sulfate;
iv. between about 30.0 g/l and about 50.0 g/l of D(+) saccharose, and
v. water.

8. The composition of claim 6, wherein the fungi and yeast are selected from the group consisting of Basillus Subtilis, Candida Albicans, Saccharomyces Cerevisiae, Saccharomyces Uvarum, Rhodotorula Rubra, Penicillium Camemberti, Aspergillus Niger, Trychophyton Ajelloi and Geotrichum Candidum.

9. The composition of claim 6, wherein the pH of the composition is between 4.5 and 5.5.

10. The composition of claim 6, wherein the installations are food industry installations.

11. A method of preparing a culture medium comprising monobasic potassium phosphate, ammonium chloride, heptahydrate magnesium sulfate, D(+) saccharose, and water, the method comprising the steps of:
a. obtaining a solution by dissolving in ultra pure water, under agitation, the monobasic potassium phosphate, the ammonium chloride, the heptahydrate magnesium sulfate and the saccharose;
b. adjusting the pH of the solution obtained in step a. to 5.0, and
c. sterilizing the solution and conserving the solution at a temperature of 4^{a}C.

12. The method of claim 11, wherein the composition comprises:
i. between about 4.5 g/l and about 5.5 g/l of monobasic potassium phosphate;
ii. between about 0.5 g/l and about 1.5 g/l of ammonium chloride;
iii. between about 0.5 g/l and about 1.5 g/l of heptahydrate magnesium sulfate;
iv. between about 30.0 g/l and about 50.0 g/l of D(+) saccharose, and
v. water.
